(19) United States
(12) Patent Application Publication
(10) Pub. No.: US 2020/0217850 A1
(43) Pub. Date: Jul. 9, 2020

(54) HETEROGENEOUS SINGLE CELL PROFILING USING MOLECULAR BARCODING

(71) Applicant: Apton Biosystems, Inc., Pleasanton, CA (US)

(72) Inventors: Niandong LIU, San Ramon, CA (US); Norman BURNS, Pleasanton, CA (US); Manohar R. FURTADO, San Ramon, CA (US); Bryan P. STAKER, San Ramon, CA (US)

(21) Appl. No.: 16/647,461

(22) PCT Filed: Sep. 14, 2018
(86) PCT No.: PCT/US2018/051183

§ 371 Date: Mar. 13, 2020

Related U.S. Application Data
(60) Provisional application No. 62/559,223, filed on Sep. 15, 2017.

(51) Int. Cl. G01N 33/58; G01N 33/543; C12Q 1/6876

(52) U.S. Cl. CPC G01N 33/582; C12Q 1/6876; G01N 33/543

(57) ABSTRACT

Disclosed herein are methods of detecting at least one target biomolecule in at least one single cell comprising lysing the single cell or cells and performing a cell identification assay and target identification assay. Also disclosed herein are methods for preparing a sample for undergoing single cell analysis, wherein the single cell analysis comprises performing a cell identification assay and a target identification assay.

### CROSS-REFERENCE TO RELATED APPLICATIONS

This application claims priority to Provisional Application No. 62/559,223, filed Sep. 15, 2017, the disclosure of which is incorporated by reference in its entirety.

### BACKGROUND OF THE INVENTION

### Field of the invention

The invention relates to methods useful for detecting one or more target biomolecules from single cells. In certain embodiments, target biomolecules, such as DNA, RNA and proteins, can be analyzed from cells enabling multiplexed transcriptomic, genomic, and/or proteomic analysis from single cells.

### Description of the Related Art

With regards to heterogeneous cell populations, such as those found in many tumors, the generation and analysis of single cells has an increasing impact on various fields of life sciences and biomedical research. The analysis of heterogeneous cell populations in bulk is only able to provide averaged data about the population, by which important information about small, but potentially relevant, subpopulations is possibly lost in the background. Cancer development is based on a complex interrelation of mutations, selection, and clonal expansion resulting in a mosaic of cells grown out of different sub-clones within a single tumor. With current available methods, rare sub-clones are difficult to detect from studying bulk populations. In contrast, the detection of one or more target biomolecules specific to one or more of a plurality of single cells can provide very useful and more accurate detailed information which may be used for therapeutic decisions for personalized medicine. A further need for the analysis of single cells relates to very rare cells, such as circulating tumor cells, which are surrounded by billions of normal blood cells and have an increasing clinical impact.

What is needed, therefore, are improved methods, devices, and compositions for profiling cell populations with individual cell resolution, including target biomolecule populations within single cells from a population. Rapid and accurate detection of a plurality of target biomolecules, such as DNA, RNA and protein, from individual cells can provide more precise identification of sub-populations of cells, including rare cell populations, such as stem cells and tumor initiating cells (e.g., circulating tumor cells).

### SUMMARY OF THE INVENTION

Described herein are methods for profiling of target biomolecules from individual cells from samples comprising a heterogeneous population of cells. In order to achieve this, the methods comprise multiple steps including, but not limited to, separation and isolation of individual cells from a sample into individual compartments, lysing the single cells and binding unique cell identifier tags to the biomolecules from the lysed cells, distributing the tagged biomolecules from a plurality of isolated cells onto a substrate and performing at least two assays on the biomolecules: a cell identification assay to determine the cellular source (i.e., single cell of origin) of a biomolecule, and a target identification assay to determine the presence or absence of at least one biomolecule. In certain embodiments, the target identification assay comprises contacting the biomolecules from the lysed cells with target barcode probes (for example, an antibody) that preferentially bind at least one target biomolecule. For each identification assay (the cell identification assay and target identification assay), probes (either cell identifier probes or target detection probes, respectively) bound to detectable markers (i.e., fluorescent markers) are contacted with the biomolecules from the lysed cells, and the presence or absence of detectable markers at spatially separate locations on the substrate are determined. In certain embodiments, the determination of the presence or absence of detectable markers comprises performing at least N detection cycles. In certain embodiments, the identification assays comprise determining L total bits of information such that L is sufficient to produce a low error rate. In certain embodiments, the presence or absence and cellular source of target proteins, DNA and/or RNA are determined.

In an aspect, disclosed herein are methods of detecting the presence or absence of one or more target biomolecules from a single cell suspected of being present in a sample comprising obtaining a sample comprising a plurality of cells suspected of comprising one or more target biomolecules; isolating single cells from the plurality of cells into individual compartments; lysing the single cells to yield cellular material comprising a plurality of biomolecules; binding at least one cell identifier tag to the plurality of biomolecules from each single cell, wherein the cell identifier tag is unique for each isolated single cell; distributing the plurality of biomolecules from the plurality of isolated cells onto a substrate such that the plurality of biomolecules are immobilized on the substrate at spatially separate regions; performing a cell identification assay to determine the cellular source for each of the plurality of immobilized biomolecules at the spatially separate regions; and performing a target identification assay to identify the presence or absence of the one or more target biomolecules at the spatially separate regions. In certain embodiments, performing the cell identification assay comprises: contacting the substrate comprising the immobilized plurality of biomolecules with a cell identifier probe set, wherein the cell identifier probe set comprises a plurality of cell identifier probes comprising a cell identification detectable marker, wherein each cell identifier probe binds preferentially to at least one cell identifier tag specific for each of the isolated single cells; removing unbound cell identifier probes from the surface of the substrate; and detecting the presence or absence of a signal from the cell identification detectable marker at the spatially separate regions. In certain embodiments, performing the cell identification assay comprises performing at least M detection cycles to generate a cell identification signal detection sequence for at least one of the spatially separate regions, wherein M is at least two, each cycle comprising contacting the substrate comprising the immobilized plurality of biomolecules with a cell identifier probe set, wherein the cell identifier probe set comprises a plurality of cell identifier probes comprising a cell identification detectable marker, wherein each of the cell identifier probe binds preferentially to at least one cell identifier tag specific for each of the isolated single cells; removing unbound cell identifier probes from the surface of the substrate; detecting the presence or absence of a signal from the cell identification detectable marker at the spatially separate regions; and if the cycle number is less than M, removing bound cell identifier probes from the substrate. In an embodiment, the method further comprises analyzing the cell identification signal detection sequence generated by the M cycles at at least one of the spatially separate regions to determine the cellular origin of the immobilized biomolecule. In certain embodiments, performing the target identification assay comprises contacting the substrate comprising the immobilized plurality of biomolecules with a target detection probe set, wherein the target detection probe set comprises a plurality of target detection probes that each bind preferentially to at least one of the one or more target biomolecules, the plurality of target detection probes each comprising a target identification detectable marker; removing unbound target detection probes from the surface of the substrate; and detecting the presence or absence of a signal from the target identification detectable marker at the spatially separate regions. In certain embodiments, performing the target identification assay comprises performing at least N detection cycles to generate a target identification signal detection sequence for at least one of the spatially separate regions, wherein N is at least two, each cycle comprising contacting the substrate comprising the immobilized plurality of biomolecules with a target detection probe set, wherein the target detection probe set comprises a plurality of target detection probes that each directly or indirectly bind preferentially to at least one of the one or more target biomolecules, the plurality of target detection probes each comprising a target identification detectable marker; removing unbound target detection probes from the surface of the substrate; detecting the presence or absence of a signal from the target identification detectable marker at the spatially separate regions; and if the cycle number is less than N, removing bound target detection probes from the substrate. In an aspect, the method further comprises analyzing the target identification signal detection sequence generated by the N cycles at at least one of the spatially separate regions to determine the presence or absence of the one or more target biomolecules. In certain embodiments, the methods comprise analyzing the signal from one or more of the spatially separate regions from the cell identification assay and the target identification assay to determine the presence or absence of the one or more target biomolecules in one or more of the plurality of single cells. In certain embodiments, the methods further comprise determining the presence or absence of a plurality of the one or more target biomolecules from one of the plurality of cells. In certain embodiments, the method further comprises contacting the cellular material with a target barcode probe comprising a target identification tag, wherein the target barcode probe preferentially binds at least one of the one or more target biomolecules, and wherein at least one of the target detection probes binds preferentially to the target identification tag. In an embodiment, the target barcode probe comprises an antibody. In an embodiment, binding of the target barcode probe to the target biomolecule is performed using a linker or adapter molecule. In an embodiment, the target identification tag comprises a target identifier oligonucleotide barcode. In an embodiment, the target detection probe comprises a target detection probe oligonucleotide, and wherein the target identifier oligonucleotide barcode comprises a sequence complementary to the target detection probe oligonucleotide. In an embodiment, the cell identifier probe binds specifically to one or more of the at least one cell identifier tags. In certain embodiments, the target detection probe binds specifically to one or more of the at least one target biomolecules. In certain embodiments, the cell identification assay and the target identification assay are performed sequentially at each of the spatially separate regions on the substrate. In certain embodiments, the cellular material comprises protein, DNA, RNA, or combinations thereof. In an embodiment, the cell identifier tag comprises a cell identifier oligonucleotide barcode. In certain embodiments, the cell identifier probe comprises a cell identifier probe oligonucleotide. In certain embodiments, the cell identifier probe oligonucleotide comprises a sequence complementary to the cell identifier oligonucleotide barcode. In an embodiment, the target detection probe comprises an antibody. In an embodiment, the cell identification detectable marker comprises a fluorescent tag. In certain embodiments, the target identification detectable marker comprises a fluorescent tag. In certain embodiments, the target biomolecule is protein. In an embodiment, the protein is created by ribosome display. In an embodiment, the target biomolecule is nucleic acid. In an embodiment, binding of the cell identifier tag to the plurality of biomarkers is performed using a linker or adapter molecule. In an embodiment, binding of the cell identifier tag to the plurality of biomolecules is performed by enzymatic conjugation. In certain embodiments, the cell identifier probe comprises a linker or adapter molecule bound to the cell identification detectable marker. In an embodiment, the target detection probe comprises a linker or adapter molecule bound to the target identification detectable marker. In certain embodiments, the sample comprises cells derived from an individual. In an embodiment, the cells are from tissue derived from a biopsy. In an embodiment, the biopsy is a tumor biopsy. In certain embodiments, the sample is suspected of comprising one or more cancer cells. In an embodiment, the cells are circulating cells derived from the blood or plasma of the individual. In certain embodiments, the target identification assay comprises determining L total bits of information such that L is sufficient to reduce a misidentification error rate of detection to less than 1 in 10², 1 in 10³, 1 in 10⁴, 1 in 10⁵, 1 in 10⁶, 1 in 10⁷, or 1 in 10⁸. In certain embodiments, the misidentification error rate comprises false positives, false negatives, or both. In an embodiment, the method comprises determining a quantity of the one or more target biomolecules from one or more of the plurality of cells. In an embodiment, the method comprises identifying at least one sub-population of cells, comprising at least one cell, within the sample.

In an aspect, disclosed herein is a method of preparing a sample for single cell analysis, comprising obtaining a sample comprising a plurality of cells suspected of comprising one or more target biomolecules; isolating single cells from the plurality of cells into individual compartments; lysing the single cells to yield cellular material comprising a plurality of biomolecules; and binding at least one cell identifier tag to the plurality of biomolecules from each single cell, and wherein the cell identifier tag is unique for each isolated single cell.

In an aspect, disclosed herein is a method of presence or absence of one or more target biomolecules from a single cell suspected of being present in a sample comprising obtaining a sample derived from a plurality of isolated cells suspected of comprising one or more target biomolecules, the sample comprising a plurality of biomolecules bound to at least one cell identifier tag unique for each one of the plurality of isolated cells; distributing the plurality of biomolecules from the plurality of isolated cells onto a substrate such that the plurality of biomolecules are immobilized on the substrate at spatially separate regions; performing a cell identification assay to determine the cellular source for each of the plurality of immobilized biomolecules at the spatially separate regions; and performing a target identification assay to identify the presence or absence of the one or more target biomolecules at the spatially separate regions.

In certain embodiments of any of the methods described herein, the isolation of the single cells from the plurality of cells is performed using a microfluidic device.

In certain embodiments of any of the methods described herein, the plurality of target biomolecules are pooled prior to the distributing step.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 illustrates a schematic and components of a Cell Identification Assay to detect the cellular source of a target biomolecule, according to an embodiment of the invention.

FIG. 2 illustrates a schematic and components of a Target Identification Assay using direct binding of a probe to a target to detect the identity of a target biomolecule, according to an embodiment of the invention.

FIG. 3 illustrates a schematic and components of a Target Identification Assay using a target binding probe having an oligonucleotide barcode, and a second detectable probe comprising an oligonucleotide sequence complementary to the barcode.

FIG. 4 is a flow diagram illustrating an embodiment of the methods of the invention for multiplex detection of target biomolecules from a sample comprising a heterogeneous group of cells. Individual cells are first isolated in wells of tissue culture plates (Step 1) and then lysed (Step 2). Next, cells identifier tags specific for each cell are added to the target biomolecules in each well (Step 3). The samples are then pooled (Step 4) and attached to a substrate (Step 5). The cell identification assay is then performed (Step 6) followed by the target identification assay (Step 7), and the results analyzed and the number of target biomolecules for each cell determined (Step 8)

FIG. 5 shows a scheme for attachment of cell identifier (cell ID) tags to protein and nucleic acid (NA) target biomolecules and attachment of tagged biomolecules to an epoxy-modified substrate surface, according to an embodiment of the invention.

### DETAILED DESCRIPTION

Advantages and Utility

Briefly, and as described in more detail below, described herein are methods for the detection of one or more target biomolecules from one or more isolated single cells or isolated population of cells from a sample comprising a heterogeneous population of cells. In certain embodiments, the application describes methods for detection of a plurality of distinct biomolecules (e.g., protein, DNA, RNA) in individual cells. In certain embodiments, target biomolecules, such as DNA, RNA and proteins, can be analyzed from individual cells enabling multiplexed transcriptomic, genomic, and/or proteomic analysis from the individual cells. In certain embodiments, methods are disclosed for the detection and/or quantification of biomolecules in individual cells without the need for an amplification step. In certain embodiments, the methods allows for accurate detection of target biomolecules in rare sub-populations of cells from a heterogeneous population of cells in a sample. In some embodiments, the methods and compositions disclosed herein enable rapid and accurate identification and/or quantification of rare cell types or variants from a heterogeneous population of cells.

Selected Definitions

Terms used in the claims and specification are defined as set forth below unless otherwise specified.

“Adaptor” or “linker” as used herein, refers to any molecule that can be used to indirectly attach any of the molecules or components described herein. For example, an “adaptor” or “linker” may be used to attach a target identification tag to a target barcode probe.

“Antibody” as used herein, refers to full length immunoglobulin proteins comprising heavy and light chains, as well as antigen binding fragments and chimeric antibodies. “Antibody” includes any single chain or multiple chain portion of an antibody that is capable of binding specifically to an epitope of an antigen. “Antibodies” includes monoclonal antibodies and polyclonal antibodies. “Antibodies” may be from any origin or any species.

“Analyte” as used herein, refers to any molecule that can be or that is intended to be detected by the methods of the invention. For example, in certain embodiments, a target biomolecule bound to a target detection probe and/or a cell identifier probe is an “analyte”.

“Barcode”, “barcode sequence” or “barcode moiety” as used herein, refers to a molecular substance that can be used to identify one or more molecules from a plurality of molecules. Barcodes can be bound, conjugated or hybridized to any target biomolecule (such as protein, DNA or RNA) directly or indirectly. In certain preferred embodiments, the barcode is a nucleotide sequence that can identify one or more nucleic acids. In certain embodiments, the barcode is a nucleotide sequence between 30 and 20 nucleotides in length, between 25 and 20 nucleotides in length, between 20 and 15 nucleotides in length, between 15 and 10 nucleotides in length or between 10 and 5 nucleotides in length. In certain embodiments, the barcode is DNA. In certain aspects, the barcode is an oligonucleotide barcode. Oligonucleotide barcodes can further comprise non-nucleic acid substances (e.g., substances used as detectable markers, etc.). As used herein, oligonucleotide sequences bound or annealed to probes, tags and binding agents can be referred to as “barcode”, “barcode sequence” or “barcode moiety”. Methods for generating oligonucleotide barcodes and methods for conjugating oligonucleotide barcodes to proteins (such as antibodies) can be performed by any method known in the art. For example, methods for conjugating barcodes to proteins are described in Kozlov et al., “Efficient strategies for the conjugation of oligonucleotides to antibodies enabling highly sensitive protein detection”; Biopolymers; 73(5); Apr. 5, 2004; pp. 621-630. Methods for generating DNA barcodes proteins by ribosome display are described, for example, in Gu et al., “Multiplex single-molecule interaction profiling of DNA barcoded proteins”; Nature. 515 (7528) Nov. 27, 2014; pp. 554-557.

“Binding” as used herein, refers to any interaction between molecules, either direct or indirect. “Binding” may be specific or non-specific. “Binding” may occur between molecules of the same type (e.g., nucleic acid binding to nucleic acid) or between molecules of different types (e.g., protein binding to nucleic acid). “Binding” may occur with any effective dissociation constant. Methods to determine specific or preferential binding are well known in the art. A molecule exhibits “specific binding” or “preferential binding” if it reacts or associates more frequently, more rapidly, with greater duration and/or with greater affinity with a particular cell or substance than it does with alternative cells or substances. For example, an antibody “specifically binds” or “preferentially binds” to a target if it binds with greater affinity, avidity, more readily, and/or with greater duration than it binds to other substances. For example, an antibody that specifically or preferentially binds to a conformational epitope of a protein target biomolecule is an antibody that binds this epitope with greater affinity, avidity, more readily, and/or with greater duration than it binds to other epitopes on the same target biomolecule or epitopes on different target biomolecules. It is also understood by reading this definition that, for example, an antibody (or moiety or epitope) that specifically or preferentially binds to a first target biomolecule may or may not specifically or preferentially bind to a second target biomolecule. As such, “binding”, “specific binding” or “preferential binding” does not necessarily require (although it can include) exclusive binding.

“Binding agent” or “target barcode probe” as used herein, refers to any molecule that can bind to a target biomolecule. In certain aspects, the binding agent is an antibody. In certain aspects, the binding agent is an oligonucleotide.

“Cycle” is defined by completion of one or more passes and stripping of the probes from the substrate, if needed, for subsequent cycles. Subsequent cycles of one or more passes per cycle can be performed. Multiple cycles can be performed on a single substrate or sample. For proteins and nucleic acids, multiple cycles will require that the probe removal (stripping) conditions. In preferred embodiments, the stripping occurs under denaturing conditions. In certain embodiments, probes for proteins are chosen to bind to peptide sequences so that the binding efficiency is independent of the protein fold configuration. Alternatively, stripping conditions can maintain proteins folded in their proper configuration.

“Bit” as used herein refers to a basic unit of information in computing and digital communications. A bit can have only one of two values. The most common representations of these values are 0 and 1. The term bit is a contraction of binary digit. In one example, a system that uses 4 bits of information can create 16 different values. All single digit hexadecimal numbers can be written with 4 bits. Binary-coded decimal is a digital encoding method for numbers using decimal notation, with each decimal digit represented by four bits. In another example, a calculation using 8 bits, there are 2⁸ (or 256) possible values.

“Detectable marker” as used herein, refers to a molecule capable of producing a signal for detecting a target biomolecule. The marker can be, but is not limited to, a fluorescent marker. The marker can comprise, but is not limited to, a fluorescent molecule, chemiluminescent molecule, chromophore, enzyme, enzyme substrate, enzyme cofactor, enzyme inhibitor, dye, metal ion, metal sol, ligand (e.g., biotin, avidin, streptavidin or haptens), radioactive isotope, markers for electrical detection (e.g., ISFET detection), markers that produce a change in pH upon a subsequent reaction, and the like. A detectable marker may comprise a plurality or a combination of markers.

“Detection” as used herein, refers to the identification of a signal produced by the methods described herein. “Detection” may or may not comprise one or more analysis steps. “Detection” as used herein, may comprise performing any method known to one of ordinary skill in the art to identify the target molecule from the signal produced by the methods described herein. For example, in certain embodiments, “detection” may comprise use of sequencing methods known in the art and/or microscopy or other imaging methods. “Detection” includes optical detection or electrical detection.

“Hybridizing” as used herein, refers to the annealing of a nucleic acid molecule to another nucleic acid molecule through the formation of one or more hydrogen bonds (e.g., base pairing of complementary nucleotides by hydrogen bond formation). Nucleic acids may be hybridized under any conditions known and used in the art to efficiently anneal oligonucleotides to nucleic acids of interest. Oligonucleotides may be hybridized in conditions that vary significantly in stringency to compensate for binding activity with respect to target binding and off-target binding.

“Lysing” as used herein, refers to the exposure of cellular contents comprising target biomolecules by disruption, permeabilization, and/or fragmentation of the plasma membrane and/or subcellular membranes (e.g., nuclear membrane). “Lysing” may occur by any means known in the art for example, by chemical (e.g., detergents) or physical means (e.g., sonication).

“Microfluidic device” as used herein, refers to and device that is used to regulate the control of movement of fluid comprising target biomolecules in low volumes (typically less than a milliliter) of fluid and can include use of droplets, substrates or other methods known in the art to direct the movement of biomolecules.

“Pass” as used herein, refers to a process where a plurality of probes or tags are introduced to the bound analytes or target biomolecules, selective binding occurs between the probes and distinct target biomolecules, and a plurality of signals are detected from the probes. In some embodiments, a pass includes introduction of a set of antibodies that bind specifically to a target analyte or target biomolecule. There can be multiple passes of different sets of probes before the substrate is stripped of all probes.

“Probe” as used herein, refers to a molecule that is capable of binding to other molecules (e.g., oligonucleotides comprising DNA or RNA, polypeptides or full-length proteins, etc.). The probe comprises a structure or component that binds directly or indirectly to the target biomolecule. In some embodiments, multiple probes may recognize different parts of the same target analyte or target biomolecule. Examples of probes include, but are not limited to, an aptamer, an antibody, a polypeptide, an oligonucleotide (DNA, RNA), or any combination thereof. In certain aspects, probes comprise a detectable label or tag. In certain aspects, probes are modified for conjugation of a detection moiety or a substrate binding moiety. In certain aspects, oligonucleotide probes are modified with a peptide nucleic acid (PNA) to block binding of a label for optimization of detection methods to account for different binding activities of probes. Probes can have a cross-reactivity with non-target sequences. In certain aspects, probes have a cross-reactivity with non-target biomolecules of greater than 2%, 5%, 10%, 15%, 20%, 25%, 50% or 75%. In general, the affinity of an oligonucleotide probe to a target oligonucleotide sequence increases continuously with oligonucleotide length. In a preferred embodiment, oligonucleotide probes have a dissociation constant in the range of about 10⁻⁹ to 10⁻⁶ molar, in the range of 10⁻⁹ to 10⁻⁸ molar, in the range of 10⁻⁸ to 10⁻⁷ or the range of 10⁻⁷ to 10⁻¹¹ molar. In certain embodiments, antibody probes bind to target biomolecules with a dissociation constant in the range of about 10⁻¹³ to 10⁻⁶ molar, in the range of 10⁻¹² to 10⁻⁷ molar, in the range of 10⁻¹¹ to 10⁻⁸ or the range of 10⁻¹⁰ to 10⁻⁹ molar.

“Sample” as used herein, refers to a specimen, culture, or collection from a biological material. Samples may be derived from or taken from a mammal, including, but not limited to, humans, monkey, rat, or mice. Samples may include materials including, but not limited to, cultures, blood, blood plasma, tissue, formalin-fixed paraffin embedded (FFPE) tissue, saliva, hair, feces, urine, and the like. These examples are not to be construed as limiting the sample types applicable to the present invention.

“Substrate” as used herein, refers to any solid or semi-solid support used for adhering to analytes (i.e., nucleic acids) of interest. A substrate can be made of any suitable material, such as, but not limited to, glass, metal, plastic, membranes, a gel, silicon, carbohydrate surfaces, etc. A substrate can be flat two-dimensional surfaces or three-dimensional surfaces, such as micro-beads or micro-spheres. In certain embodiments, the substrate is configured for electrical detection methods described herein. In certain embodiments, a substrate comprises one or more ISFETs. A substrate can be an integrated-circuit chip that contains one or more ISFETs. Substrates can be coated or treated with substances to alter the binding characteristics of the substrate to biomolecules of interest (e.g., glass or silicon surfaces treated with amino silane and glass surfaces treated with epoxy silane-derivatized or isothiocyanate). Substrates may also be coated or bound to adapters (such as oligonucleotides) that specifically bind targets of interest (e.g., the enriched nucleic acid, ligation products and amplification products). Adapters, including oligonucleotide adapters coated on substrates can be used to generate addressable arrays wherein the location of the oligonucleotide adapters at distinct regions on the substrate correspond to specific targets.

“Sufficient amount” means an amount sufficient to produce a desired effect, e.g., an amount sufficient to detect a target biomolecule in a single cell.

“Target biomolecule” refers to as used herein refers to a molecule, compound, substance or component that is desired to be identified, quantified, or otherwise characterized. A target biomolecule can comprise by way of example, but not limitation, an atom, a compound, a molecule (of any molecular size), a polypeptide, a protein (folded or unfolded), an oligonucleotide molecule (RNA, cDNA, or DNA), a fragment thereof, a modified molecule thereof, such as a modified nucleic acid, or a combination thereof. Generally, a target biomolecule can be at any of a wide range of concentrations (e.g., from the mg/mL to ag/mL range), in any volume of solution (e.g., as low as the picoliter range). For example, samples of blood, serum, formalin-fixed paraffin embedded (FFPE) tissue, saliva, or urine could contain various target biomolecules. The target biomolecules are recognized by probes, which are used to identify and quantify the target biomolecules using electrical or optical detection methods.

Abbreviations used in this application include the following: “DNA” deoxyribonucleic acid, “RNA” ribonucleic acid and “ISFET” ion-sensitive field-effect transistor.

It must be noted that, as used in the specification and the appended claims, the singular forms “a,” “an” and “the” include plural referents unless the context clearly dictates otherwise.

Methods of the Invention

Described herein are methods useful for detecting one or more target biomolecules from at least one single cell. The methods comprise multiple steps including, but not limited to separation and isolation of individual cells from a sample into individual compartments, lysing the single cells and binding unique cell identifier tags to the biomolecules from the lysed cells, distributing the tagged biomolecules from a plurality of isolated cells onto a substrate and performing at least two assays on the biomolecules: a cell identification assay to determine the cellular source (i.e., single cell of origin) of a biomolecule, and a target identification assay to determine the identity and presence or absence of at least one biomolecule.

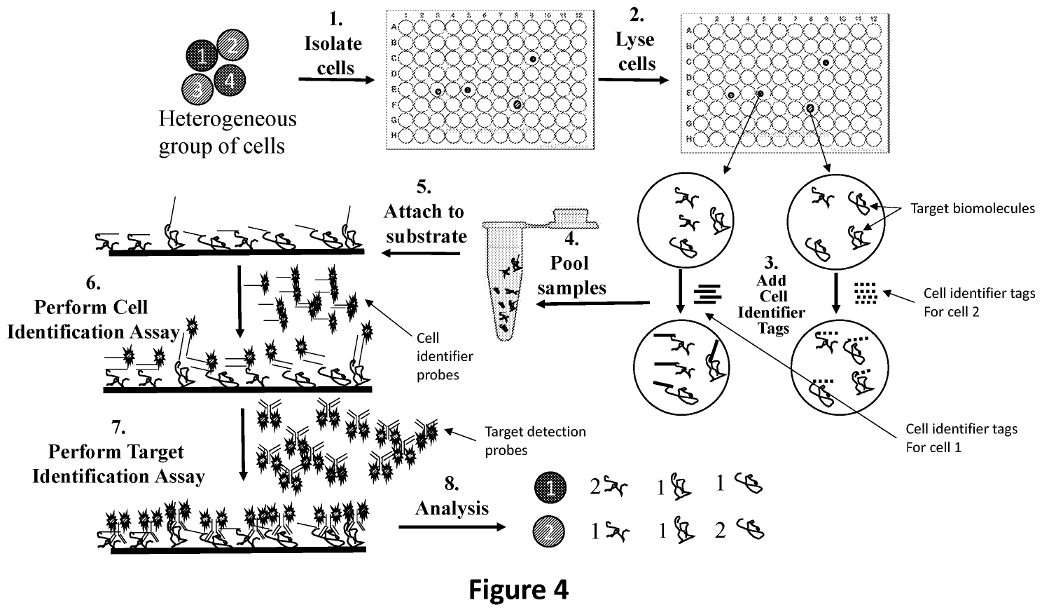

FIG. 4 illustrates steps generally performed according to an embodiment of the invention, where individual cells are first isolated in wells of tissue culture plates (Step 1) and then lysed (Step 2). Next, cells identifier tags specific for each cell are added to the target biomolecules in each well (Step 3). The samples are then pooled (Step 4) and attached to a substrate (Step 5). The cell identification assay is then performed (Step 6) followed by the target identification assay (Step 7), and the results analyzed and the number of target biomolecules for each cell determined (Step 8).

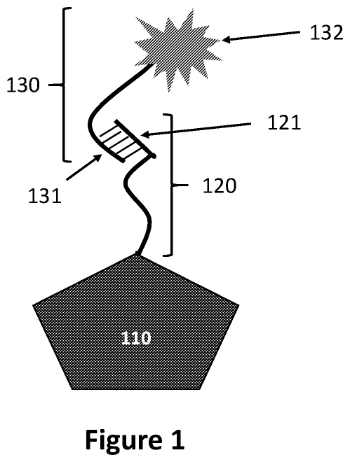

FIG. 1 illustrates a complex formed during an embodiment of a Cell Identification Assay. When isolated into a compartment, a single cell or population of cells is lysed and the biomolecules 110 from the single cell or population of cells are tagged with a cell identifier tag 120 comprising a cell identifier oligonucleotide barcode 121. The biomolecules from multiple compartments from multiple isolated cells or cell populations can then be distributed onto a surface for single molecule identification, where an assay to detect their cellular origin can be performed. In the embodiment shown in FIG. 1, the biomolecules are contacted with a set of cell identifier probes 130 comprising a cell identifier probe oligonucleotide 131 and a cell identification detectable marker 132. The cell identifier oligonucleotide barcode 121 is hybridized to a matching or complementary cell identifier probe oligonucleotide 131 on a cell identifier probe 130. After washing to remove unbound probes, a signal generated by the cell identification detectable marker 132 can be read to facilitate detection of the cellular origin of the target biomolecule 110.

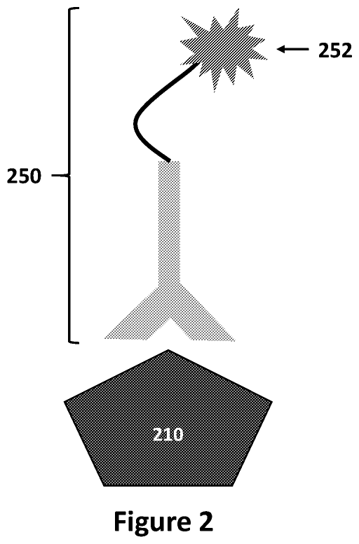

FIG. 2 illustrates a complex formed during an embodiment of a Target Identification Assay. After target biomolecules 210 are distributed onto a surface for single molecule detection, a target detection probe set comprising probes (for example, an antibody) that bind specifically to at least one target biomolecule can be mixed with the target biomolecules. As shown, each target detection probe 250 is directly bound to the target biomolecule 210. The probe comprises a target identification detectable marker 252. After washing to remove unbound probe, a signal generated by the target identification detectable marker 252 can be read to facilitate detection of the identity of the target biomolecule.

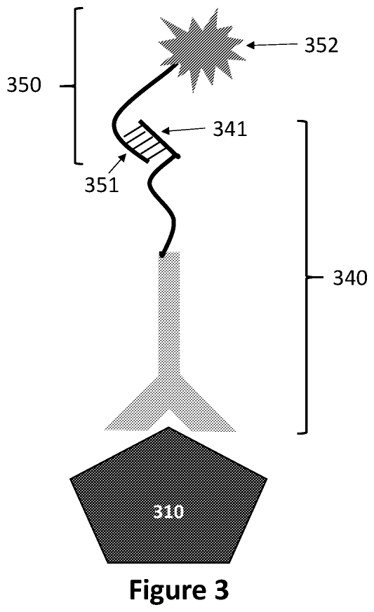

In certain embodiments, the target identification assay further comprises contacting the biomolecules from the lysed cells with target barcode probes (for example, a barcoded antibody) that preferentially bind at least one target biomolecule. FIG. 3 illustrates complex formed during an embodiment of the Target Identification Assay using 2 probe sets, a first set of probes containing target barcode probes 340 that bind specifically to at least one target biomolecule 310 and include a target identifier oligonucleotide barcode 341 specific to the identity of the target biomolecule 310 bound by the probe, and a second set of probes containing target detection probes 350 that have a target detection probe oligonucleotide 351 that binds specifically to the target identifier oligonucleotide barcode 341 and comprises a target identification detectable marker 352. In this embodiment, after target biomolecules 310 are distributed onto a surface for single molecule detection, the target barcode probe set comprising target barcode probes 340 that bind specifically to at least one target biomolecule 310 can be mixed with the target biomolecules. As shown in FIG. 3, a target detection probe 340 binds specifically to its respective target biomolecule 310. The target detection probe comprises a target identifier oligonucleotide barcode 341. After optionally washing to remove unbound target barcode probes, the second probe set, i.e., the set of target detection probes 350 can be mixed with the target biomolecules. As shown in FIG. 3, the target detection probe 350 hybridizes specifically to its respective target detection probe via a target detection probe oligonucleotide 351 having a sequence complementary to its respective target identifier oligonucleotide barcode 341. After washing to remove unbound probe, a signal generated by the target identification detectable marker 352 can be read to facilitate detection of the identity of the target biomolecule 310.

In some embodiments, the target identifier oligonucleotide barcode 341 and target detection probe oligonucleotide comprise at least 5, 6, 7, 8, 9 10, 15, 20, 25, 30, 35, 40, 45, or 50 nucleotides that are complementary to each other to facilitate hybridization.

For each identification assay (the Cell Identification Assay and Target Identification Assay), probes (either cell identifier probes or target detection probes, respectively) bound to detectable markers (e.g., fluorescent markers) are contacted with the biomolecules from the lysed cells, and the presence or absence of detectable markers at spatially separate locations on the substrate are determined.

As described further herein, each identification step can be performed multiple times using different sets of probes to generate additional information to reduce false positive or false negative single molecule detection error rates. Thus, not all probes must be specific for a single target, but instead are specific for a subset of targets, as redundancy introduced from cycled detection with multiple probe sets can still generate information to reliably and repeatedly detect the identity and cellular origin of each target biomolecule.

In certain embodiments, the determination of the presence or absence of detectable markers comprises performing at least N detection cycles. In certain embodiments, the presence or absence and cellular source of target proteins, DNA and/or RNA are determined.

Cell Identification Assay

The methods described herein comprise performing a cell identification assay to determine the cellular source of a target biomolecule. The cell identification assay comprises contacting the substrate comprising an immobilized plurality of biomolecules with a cell identifier probe set, wherein the cell identifier probe set comprises a plurality of cell identifier probes comprising a cell identification detectable marker, wherein each cell identifier probe binds preferentially to at least one cell identifier tag specific for each isolated single cells. In certain embodiments, the cell identification assay comprises the step of removing unbound cell identifier probes from the surface of the substrate; and detecting the presence or absence of a signal from the cell identification detectable marker at the spatially separate regions on the substrate.

In certain embodiments, the cell identification assay comprises performing at least M detection cycles to generate a cell identification signal detection sequence for at least one of the spatially separate regions, wherein M is at least two, each cycle comprising contacting the substrate comprising the immobilized plurality of biomolecules with a cell identifier probe set, wherein the cell identifier probe set comprises a plurality of cell identifier probes comprising a cell identification detectable marker, wherein each of the cell identifier probe binds preferentially to at least one cell identifier tag specific for each of the isolated single cells; removing unbound cell identifier probes from the surface of the substrate; detecting the presence or absence of a signal from the cell identification detectable marker at the spatially separate regions; and if the cycle number is less than M, removing bound cell identifier probes from the substrate.

In certain embodiments, M is greater than 1, 2, 3, 4, 5, 10, 15, 20, 25, 30, 35, 40, 45, or 50. In an aspect, M is sufficient to detect a target biomolecule with a false positive detection rate of less than 1 in 10⁶.

Isolation and Lysis of Single Cells

Described herein are methods comprising the step of isolation of individual cells from a sample, wherein the cells are separated and isolated into individual compartments. The methods used to separate cells will depend, in part, on the origin and type of sample being used. For example separation of individual cells from blood or single cell suspension of tissue can be performed by methods routinely performed in the art, such as flow cytometry or microfluidic techniques (e.g., single-cell sorting using fluorescence-activated cell sorting (FACS) techniques).

In certain embodiments, single cells obtained or separated from tissue are isolated into individual compartments, for example, by placement into individual wells of a tissue culture plate or in microfluidic droplets. In certain embodiments, the individual cells are encapsulated in individual gel beads. In certain aspects, the beads are plastic, glass, silica or metallic and the target biomolecules are released from the beads by a chemical or enzymatic reaction.

In certain embodiments, individual cells are encapsulated in individual oil droplets. In some embodiments, the oil droplets are aqueous solutions surrounded by oil. In certain embodiments, the oil is immiscible with water. In certain embodiments, the oil is transparent. In certain embodiments, the oil droplet has a volume of 1 pL to 100 nL. In certain embodiments, an aqueous solution surrounded by oil comprises buffer solutions. In certain embodiments, a surfactant is added to the oil droplets.

The methods comprise lysis of individual cells to expose target biomolecules for detection. The protocol for lysis of cells depends, in part, upon the nature and sub-cellular location of the target biomolecules to be detected. Any method known in the art for the lysis of membranes and/or extraction of target biomolecules from cells may be employed. Examples of lysis agents include, but are not limited to detergents (e.g., NP-40 (nonyl phenoxypolyethoxylethanol)), surfactants (e.g., non-ionic surfactant such as TritonX-100 and Tween 20, or ionic surfactants such as sarcosyl and sodium dodecyl sulfate), or lysis enzymes (e.g. lysozyme). In certain embodiments, the lysis agents disrupt cellular membranes but do not disrupt oil droplets. In other embodiments, non-reagent based lysis systems can be used including, but not limited to, heat, electroporation, mechanical disruption, and acoustic disruption (e.g., sonication). In an embodiment, the cells are lysed with a solution comprising at least one detergent, surfactant, or lysis enzyme. In certain embodiments, the cells are lysed using a combination of lysis reagents and techniques. In certain embodiments, the surfactant is Triton X-100. In another embodiment, the detergent is NP-40 (nonyl phenoxypolyethoxylethanol). In an embodiment, the cells are lysed with a buffer comprising sodium dodecyl sulfate. In certain embodiments, the cellular material released from the lysed cells comprises cellular proteins. In certain aspects, the lysis of cells is performed in individual single cell compartments.

In certain embodiments, the RNA, DNA and proteins from cells can be separately extracted from individual cells enabling multiplexed transcriptomic, genomic, and/or proteomic analysis from each cell. In an aspect, the RNA, DNA and proteins can be extracted using an extraction reagent that allows for simultaneous isolation of RNA, DNA and protein.

Cell Identifier Tags

Cell identifier tags can be bound either directly or indirectly to a plurality of target biomolecules. In an aspect, cell identifier tags are designed to also bind directly or indirectly, to a corresponding cell identifier probe. In certain aspects, the cell identifier tag comprises nucleic acid. In certain embodiments, the cell identifier tag is an oligonucleotide (FIG. 1). In certain aspects, the cell identifier tag is conjugated directly to a target biomolecule. In certain aspects, the cell identifier tag is conjugated to a target biomolecule indirectly using an adaptor or linker.

In certain embodiments, the cell identifier tag is an oligonucleotide between 2 and 50 nucleotides in length. In certain embodiments, the cell identifier tag is between 2 and 10, 10 and 20, 20 and 30, 30 and 40 or 40 and 50 nucleotides in length.

In certain embodiments, the cell identifier tag is an oligonucleotide that has been created by a method comprising ribosome display. In certain embodiments the cell identifier tag comprises a cell identifier oligonucleotide barcode. Methods for generating proteins bound to oligonucleotide barcodes are described, for example, in Gu et al., Nature 515 (7528) 2014.

Cell identifier oligonucleotide barcodes may be any length that allows efficient binding to a target sequence. In certain aspects, the cell identifier oligonucleotide barcodes are less than 200 nucleotides in length, less than 100 nucleotides in length, less than 80 nucleotides in length, less than 50 nucleotides in length, less than 40 nucleotides in length, less than 30 nucleotides in length or less than 20 nucleotides in length. The complementarity of the cell identifier oligonucleotide barcodes to the cell identifier probe oligonucleotide is a precise pairing such that stable and specific binding occurs between nucleic acid sequences e.g., between a cell identifier probe oligonucleotide sequence and the cell identifier oligonucleotide barcode sequence (e.g., nucleotide sequence variant) of interest. It is understood that the sequence of a nucleic acid need not be 100% complementary to that of its target or complement. In some cases, the sequence is complementary to the other sequence with the exception of 1-2 mismatches. In some cases, the sequences are complementary except for 1 mismatch. In some cases, the sequences are complementary except for 2 mismatches. In some cases, the sequences are complementary except for 3 mismatches. In yet other cases, the sequences are complementary except for 4, 5, 6, 7, 8, 9 or more mismatches. In certain aspects, the number of mismatches is 20% or less, 10% or less, 5% or less or 2% or less of the number of nucleotides present in the cell identifier oligonucleotide barcode. In certain aspects, the cell identifier oligonucleotide barcode and the cell identifier probe oligonucleotide are complementary to at least 18, at least 17, at least 16, at least 15, at least 14, at least 13, at least 12, at least 11, at least 1, at least 9, at least 8, at least 7, at least 6 or at least 5 nucleotides of a target nucleotide sequence. In certain aspects, tags are complementary to one or more individual probes. In certain aspects, the tags do not bind to alternative sequences because of mismatches in sequences leading to loss of complementarity.

In certain embodiments, cell identifier tags are conjugated or bound to target biomolecules using enzymatic conjugation.

Cell Identifier Probes

Cell identifier probes can be bound either directly or indirectly to a cell identification detectable marker. In an aspect, cell identifier tags are designed to also bind directly or indirectly, a corresponding cell identifier tag. In certain aspects, the cell identifier probe comprises nucleic acid. In certain embodiments, the cell identifier probe is an oligonucleotide. In certain aspects, the cell identifier probe is conjugated directly to a cell identification detectable marker. In certain aspects, the cell identifier probe is conjugated to a cell identification detectable marker indirectly using an adaptor or linker.

In some embodiments, between 2 and 50 different cell identifier probes comprise a cell identifier probe set, wherein each type of cell identifier probe detects a distinct cell identifier tag. In certain embodiments, between 50 and 100, between 100 and 200, between 200 and 300, between 300 and 400, between 400 and 500, between 500 and 1,000, or between 1,000 and 10,000 distinct cell identifier probes are in a cell identifier probe set.

Cell identifier probe oligonucleotides may be any length that allows efficient binding to a tag sequence. In certain embodiments, the cell identifier probe oligonucleotides are less than 200 nucleotides in length, less than 100 nucleotides in length, less than 80 nucleotides in length, less than 50 nucleotides in length, less than 40 nucleotides in length, less than 30 nucleotides in length or less than 20 nucleotides in length. The complementarity of the cell identifier probe oligonucleotides to the cell identifier oligonucleotide barcode is a precise pairing such that stable and specific binding occurs between nucleic acid sequences e.g., between a probe sequence and the barcode sequence (e.g., nucleotide sequence variant) of interest. It is understood that the sequence of a nucleic acid need not be 100% complementary to that of its target or complement. In some cases, the sequence is complementary to the other sequence with the exception of 1-2 mismatches. In some cases, the sequences are complementary except for 1 mismatch. In some cases, the sequences are complementary except for 2 mismatches. In other cases, the sequences are complementary except for 3 mismatches. In yet other cases, the sequences are complementary except for 4, 5, 6, 7, 8, 9 or more mismatches. In certain aspects, the number of mismatches is 20% or less, 10% or less, 5% or less or 2% or less of the number of nucleotides present in the cell identifier probe oligonucleotide. In certain aspects, the cell identifier probe oligonucleotide and cell identifier oligonucleotide barcode are complementary to at least 18, at least 17, at least 16, at least 15, at least 14, at least 13, at least 12, at least 11, at least 1, at least 9, at least 8, at least 7, at least 6 or at least 5 nucleotides of a target nucleotide sequence. In certain aspects, cell identifier probe oligonucleotides are complementary to one or more individual cell identifier oligonucleotide barcodes. In certain aspects, the cell identifier probe oligonucleotides do not bind to alternative sequences because of mismatches in sequences leading to loss of complementarity.

Cell Identification Detectable Marker

In some embodiments, the cell identification detectable marker can be any molecule capable of producing a signal for detecting a target biomolecule. For example, the cell identification detectable marker can be a fluorescent marker. The cell identification detectable marker can comprise, but is not limited to, a fluorescent molecule, chemiluminescent molecule, chromophore, enzyme, enzyme substrate, enzyme cofactor, enzyme inhibitor, dye, metal ion, metal sol, ligand (e.g., biotin, avidin, streptavidin or haptens), radioactive isotope, molecules designed for electronic/ionic detection (e.g., by ISFETs) and the like, and combinations thereof.

Detectable markers can be attached chemically and/or covalently to any appropriate region of the cell identifier probe. In some embodiments, the detectable markers are fluorescent molecules. Fluorescent molecules can be fluorescent proteins or can be a reactive derivative of a fluorescent molecule known as a fluorophore. Fluorophores are fluorescent chemical compounds that emit light upon light excitation. In some embodiments, the fluorophore selectively binds to a specific region or functional group on the target molecule and can be attached chemically or biologically. Examples of fluorescent tags include, but are not limited to, green fluorescent protein (GFP), yellow fluorescent protein (YFP), red fluorescent protein (RFP), cyan fluorescent protein (CFP), fluorescein, fluorescein isothiocyanate (FITC), tetramethylrhodamine isothiocyanate (TRITC), cyanine (Cy3), phycoerythrin (R-PE) 5,6-carboxymethyl fluorescein, (5-carboxyfluorescein-N-hydroxysuccinimide ester), Texas red, nitrobenz-2-oxa-1,3-diazol-4-yl (NBD), coumarin, dansyl chloride, and rhodamine (5,6-tetramethyl rhodamine).

In certain embodiments the detection markers are configured for electronic detection. For example, the detectable marker can release ions upon a subsequent reaction, changing the pH of its environment in a manner that is reliably detectable.

Target Identification Assay

The methods described herein comprise performing a target identification assay to identify one or more distinct target biomolecules from individual cells. The target identification assay comprises contacting a substrate comprising an immobilized plurality of biomolecules with a target detection probe set, wherein the target detection probe set comprises a plurality of target detection probes that each bind preferentially to at least one of said one or more target biomolecules. The target detection probes each comprise a target identification detectable marker. Unbound target detection probes are removed from the surface of the substrate. The presence or absence of a signal is determined from the target identification detectable marker at spatially separate regions on the substrate.

In certain embodiments, the target identification assay comprises performing at least N detection cycles to generate a target identification signal detection sequence for at least one of the spatially separate regions on the substrate. In certain embodiments, N is at least two, and each cycle comprises contacting the substrate comprising the immobilized plurality of biomolecules with a target detection probe set. The target detection probe set comprises a plurality of target detection probes that each directly or indirectly bind preferentially to at least one of the one or more target biomolecules, and the plurality of target detection probes each comprise a target identification detectable marker. The target identification assay further comprises the step of removing unbound target detection probes from the surface of the substrate; detecting the presence or absence of a signal from the target identification detectable marker at the spatially separate regions; and if the cycle number is less than N, removing bound target detection probes from the substrate.

Target Detection Probes

In certain embodiments, the target identification assay comprises contacting the cellular material suspected of containing target biomolecules with target detection probes, wherein the target detection probes comprise a target identification detectable marker. In some embodiments, the target detection probe binds directly to a target biomolecule (FIG. 2).

In certain embodiments, the target detection probe comprises an oligonucleotide (target detection probe oligonucleotide). In certain embodiments, a target detection probe, comprising a target identification detectable marker and a target detection probe oligonucleotide, is bound indirectly to a target barcode probe by annealing of the target detection probe oligonucleotide to the target identifier oligonucleotide barcode (FIG. 3).

In certain embodiments, the target detection probe is complementary to a target biomolecule that is a nucleic acid. In certain aspects, the target biomolecule is DNA. In certain other aspects, the target biomolecule is mRNA.

In certain embodiments, the target detection probe is an oligonucleotide that has been created by a process comprising ribosome display. Methods for generating proteins bound to oligonucleotide barcodes by ribosome display are described, for example, in Gu et al., “Multiplex single-molecule interaction profiling of DNA barcoded proteins”; Nature. 515 (7528) Nov. 27, 2014; pp. 554-557.

Target detection probe oligonucleotides may be any length that allows efficient binding to a target sequence. In certain aspects, target detection probe oligonucleotides are less than 200 nucleotides in length, less than 100 nucleotides in length, less than 80 nucleotides in length, less than 50 nucleotides in length, less than 40 nucleotides in length, less than 30 nucleotides in length or less than 20 nucleotides in length. The complementarity of the target detection probe oligonucleotide is a precise pairing such that stable and specific binding occurs between nucleic acid sequences e.g., between a target detection probe oligonucleotide sequence and the target identifier oligonucleotide barcode sequence. It is understood that the sequence of a nucleic acid need not be 100% complementary to that of its target or complement. In some cases, the sequence is complementary to the other sequence with the exception of 1-2 mismatches. In some cases, the sequences are complementary except for 1 mismatch. In some cases, the sequences are complementary except for 2 mismatches. In some cases, the sequences are complementary except for 3 mismatches. In yet other cases, the sequences are complementary except for 4, 5, 6, 7, 8, 9 or more mismatches. In certain aspects, the number of mismatches is 20% or less, 10% or less, 5% or less or 2% or less of the number of nucleotides present in the target detection probe oligonucleotide. In certain aspects, the target detection probe oligonucleotide are complementary to at least 18, at least 17, at least 16, at least 15, at least 14, at least 13, at least 12, at least 11, at least 1, at least 9, at least 8, at least 7, at least 6 or at least 5 nucleotides of a target identifier oligonucleotide barcode sequence. In certain aspects, the target detection probe oligonucleotide are complementary to one or more individual target identifier oligonucleotide barcode sequences. In certain aspects, the target detection probe oligonucleotides do not bind to alternative sequences because of mismatches in sequences leading to loss of complementarity.

In some embodiments, between 2 and 50 different target detection probes are used, wherein each type of target detection probe detects a distinct target biomolecule. In certain embodiments, between 50 and 100, between 100 and 200, between 200 and 300, between 300 and 400, between 400 and 500, between 500 and 1,000, or between 1,000 and 10,000 distinct target detection probes are in a target detection probe set.

In certain aspects, target detection probes are conjugated or bound to target biomolecules using enzymatic conjugation.

Target Barcode Probes

In certain embodiments, the target identification assay comprises contacting cellular material from single cells with target barcode probes. In some embodiments, the target barcode probe binds a protein. In some embodiments, the target barcode probe binds nucleic acid. In an embodiment, the target barcode probe binds DNA. In an embodiment, the target barcode probe binds RNA. In some embodiments, the target barcode probe binds a sugar. In some embodiments, the target barcode probe binds a lipid. In an embodiment, the target barcode probe binds a nucleic acid. In an embodiment, the target barcode probe binds a particular covalent modification of molecules. In an embodiment, the target barcode probe comprises an antibody that binds a covalent modification of a protein. In an embodiment, the target barcode probe comprises an antibody the binds a phosphorylated amino acid on a protein. In an embodiment, the target barcode probe comprises an antibody the binds a methylated or an acetylated amino acid on a protein. In an embodiment, the target barcode probe comprises an antibody that binds a carbohydrate, lipid, acetyl group, formyl group, acyl group, SUMO protein, Ubiquitin, Nedd or Prokaryotic ubiquitin-like protein on a protein of interest.

In certain embodiments, the target barcode probe comprises a target identification tag. In certain aspects, the target identification tag is preferentially operably associated with at least one of the target biomolecules. In certain embodiments, at least one of the target detection probes binds preferentially to a target identification tag.

In certain embodiments, the target barcode probe comprises an antibody. In certain embodiments, the target barcode probe comprises a nucleic acid. In certain aspects, the target barcode probe comprises a target identifier oligonucleotide barcode. In some embodiments, the target barcode probe comprises an antibody conjugated with an oligonucleotide. In certain embodiments, the target identifier oligonucleotide barcode comprises sequences that bind preferentially to one or more target detection probe oligonucleotides (FIG. 3).

Oligonucleotides can be conjugated to antibodies by a number of methods known in the art (Kozlov et al., “Efficient strategies for the conjugation of oligonucleotides to antibodies enabling highly sensitive protein detection”; Biopolymers; 73(5); Apr. 5, 2004; pp. 621-630). Aldehydes can be introduced to antibodies by modification of primary amines or oxidation of carbohydrate residues. Aldehyde- or hydrazine-modified oligonucleotides are prepared either during phosphoramidite synthesis or by post-synthesis derivatization. Conjugation between the modified oligonucleotide and antibody result in the formation of a hydrazone bond that is stable over long periods of time under physiological conditions. Oligonucleotides can also be conjugated to antibodies by producing chemical handles through thiol/maleimide chemistry, azide/alkyne chemistry, tetrazine/cyclooctyne chemistry and other click chemistries. These chemical handles are prepared either during phosphoramidite synthesis or post-synthesis.

In some embodiments, between 2 and 50 different target barcode probes are used in a target identification assay, wherein each type of target barcode probe detects a distinct target biomolecule. In certain embodiments, between 50 and 100, between 100 and 200, between 200 and 300, between 300 and 400, between 400 and 500, between 500 and 1,000, or between 1,000 and 10,000 distinct target barcode probes are used in a target identification assay.

Target Identification Detectable Marker

In certain embodiments, the target identification detectable marker can be any molecule capable of producing a signal for detecting a target biomolecule. For example, the target identification detectable marker can be a fluorescent marker. The target identification detectable marker can comprise, but is not limited to, a fluorescent molecule, chemiluminescent molecule, chromophore, enzyme, enzyme substrate, enzyme cofactor, enzyme inhibitor, dye, metal ion, metal sol, ligand (e.g., biotin, avidin, streptavidin or haptens), radioactive isotope, and the like, and combinations thereof.

Detectable markers can be attached chemically or covalently to any appropriate region of the target detection probe. In some embodiments, the detectable markers are fluorescent molecules. Fluorescent molecules can be fluorescent proteins or can be a reactive derivative of a fluorescent molecule known as a fluorophore. Fluorophores are fluorescent chemical compounds that emit light upon light excitation. In some embodiments, the fluorophore selectively binds to a specific region or functional group on the target molecule and can be attached chemically or biologically. Examples of fluorescent tags include, but are not limited to, green fluorescent protein (GFP), yellow fluorescent protein (YFP), red fluorescent protein (RFP), cyan fluorescent protein (CFP), fluorescein, fluorescein isothiocyanate (FITC), tetramethylrhodamine isothiocyanate (TRITC), cyanine (Cy3), phycoerythrin (R-PE) 5,6-carboxymethyl fluorescein, (5-carboxyfluorescein-N-hydroxysuccinimide ester), Texas red, nitrobenz-2-oxa-1,3-diazol-4-yl (NBD), coumarin, dansyl chloride, and rhodamine (5,6-tetramethyl rhodamine).

In certain embodiments the detection markers are configured for electronic detection.

Methods for Binding Probes and Removing Unbound Probes

In certain aspects, the cell identifier probes and/or target detection probes have a cross-reactivity with one or more non-target biomolecules of greater than 2%, 5%, 10%, 15%, 20%, or 25%. In certain aspects, at least one of the target biomolecules does not bind to a corresponding cell identifier probe and/or target detection probe for at least 10%, at least 20%, at least 30%, or at least 40% of cycles.

In certain aspects, the cell identifier probes and/or target detection probes are modified. In certain aspects, the amount of probes or the concentration of each of the cell identifier probes and/or target detection probes is optimized to account for the difference in binding affinities and cross-reactivity of the individual probes. In certain aspects, the cell identifier probes and/or target detection probes are modified with a peptide nucleic acid (PNA) to block binding of a label for optimization of detection methods to account for the different binding activities of probes.

Cell identifier probes and/or target detection probes comprising oligonucleotides may be hybridized to target biomolecules under any conditions known and used in the art to efficiently anneal oligonucleotide probes to nucleic acids of interest. Probes may be hybridized in conditions that vary significantly in stringency to compensate for probe binding activity with respect to target binding and off-target binding. Probe hybridization conditions can also vary depending on, for example, probe length, probe sequence (such as G+C content), concentration of nucleic acid present in the sample. The methods for removing or washing unbound probes will also vary significantly in stringency to compensate for probe binding activity with respect to target binding and off-target binding. Generally, more stringent conditions (such as higher temperature or use of buffers with detergents or denaturants and lower salt concentration) are used when probes are longer or have greater numbers of similar sequences present in the sample to reduce non-specific or off-target binding.

Design and Synthesis of Probes and Tags Comprising Oligonucleotides

In certain embodiments, oligonucleotides are used herein to identify a cell of origin or to detect a target biomolecule. In certain aspects, the oligonucleotide sequence determines the binding of the cell identifier probe or the target detection probe to the target biomolecule by annealing to a cell identifier tag or target identification tag (target identifier oligonucleotide barcode), respectively. In certain aspects, oligonucleotide probes comprise a barcode. In certain aspects, an oligonucleotide probe comprises more than one barcode. In certain embodiments, the barcode is a nucleotide sequence between 30 and 20 nucleotides in length, between 25 and 20 nucleotides in length, between 20 and 15 nucleotides in length, between 15 and 10 nucleotides in length or between 10 and 5 nucleotides in length. In certain embodiments, the barcode is DNA. Barcodes can further comprise non-nucleic acid substances (e.g., substances used as tags, etc.).

Methods for the synthesis of barcodes include, in certain embodiments, random addition of mixed bases during nucleic acid synthesis to produce a sequence that can be used to identify a specific oligonucleotide molecule through analysis of sequencing data. In certain embodiments, synthesis of barcodes comprises the controlled addition of bases to generate a known sequence. In certain embodiments, barcode sequences can be verified by sequencing. In certain aspects, barcodes can be synthesized and extended using polymerase to attach the barcode to oligonucleotides on probes and tags such as, cell identifier probes, target detection probes, cell identifier tags and target identification tags. In other aspects, barcode sequences can be synthesized without probes and either ligated or annealed to the probes in a separate step.

Distribution and Immobilitzation of Target Biomolecules to Substrates

Target biomolecules, probes, tags, agents and oligonucleotides described herein can comprise substrate binding moieties for immobilization and/or binding of the target biomolecule to the substrate. The nature of the substrate binding moieties will correspond to the type of substrate or solid support to be used for binding to the target biomolecule. A substrate can be any solid or semi-solid support used for adhering to analytes/target biomolecules. A substrate can be made of any suitable material, such as, but not limited to, glass, metal, plastic, a gel, membranes, silicon, a carbohydrate surface, etc. Substrate binding moieties can be, for examples, modified nucleotides. Proteins and/or oligonucleotides can be modified by any suitable method known in the art for attachment and/or immobilization of protein and/or nucleic acid to substrates, for example, by conjugation to biotin, generating amine or thiol group modifications, covalent linkage to a thioester or conjugation to a cholesterol-TEG. Modification of oligonucleotides to produce substrate binding moieties may occur at the 5′ terminus, 3′ terminus or at any position within the oligonucleotide. Linkers or spacers may be added between the terminus of the oligonucleotide and the substrate binding moiety. Substrate binding moieties may be bound directly or indirectly to the target biomolecules, probes, tags, agents and oligonucleotides described herein.

The type of solid support chosen can be chosen based on: the level of scattering and fluorescence background inherent in the support material and added chemical groups; the chemical stability and complexity of the construct; the amenability to chemical modification or derivatization; surface area; loading capacity and the degree of non-specific binding of the final product. Substrates can be prepared by treating glass or silicon surfaces, for example, with avidin for the binding to biotin-conjugated oligonucleotides. In another example, glass or silicon surfaces can be treated with an amino silane. Oligonucleotides modified with an NH2 group can be immobilized onto epoxy silane-derivatized or isothiocyanate coated glass slides. Succinylated oligonucleotides can be coupled to aminophenyl- or aminopropyl-derivatized glass slides by peptide bonds, and disulfide-modified oligonucleotides can be immobilized onto a mercaptosilanized glass support by a thiol/disulfide exchange reaction or through chemical cross-linkers. Amine-modified oligonucleotides can be reacted with carboxylate-modified micro-spheres with a carbodiimide, such as EDAC. Substrates may also be magnetic (such as magnetic microspheres) and bind to oligonucleotides conjugated or annealed to magnetic moieties.

Methods for Optical Detection of Target Biomoleucles

For optical detection of the target biomolecules, in certain embodiments, the target biomolecules are spatially separated on a solid substrate, so that there is no overlap of fluorescent signals. For a random array, multiple pixels are needed for each fluorescent spot. The number of pixels can be as few as 1 and as many as hundreds of pixels per spot. It is expected that the optimal amount of pixels per fluorescent spot is between 5 and 20 pixels. In one example, an imaging system has 224 nm pixels. For a system with 10 pixels per fluorescent spot on average, there is a surface density of 2 fluorescent pixels/μm². This does not mean that the surface density of the target biomolecule needs to be this low. If probes are only chosen for low abundance target biomolecules, then the amount of target biomolecules on the surface may be much higher. For instance, if there are, on average, 20,000 target biomolecules per μm² on the surface, and probes are chosen only for the rarest 0.01% (as an integrated sum) target biomolecule, then the fluorescent analyte/target biomolecule surface density will be 2 fluorescent pixels/μm². In an embodiment, the imaging system has 163 nm pixels. In an embodiment, the imaging system has 224 nm pixels. In a preferred embodiment, the imaging system has 325 nm pixels. In other embodiments, the imaging system has as large as 500 nm pixels.

Optical detection methods can be used to quantify and identify a large number of target biomolecules simultaneously in a sample. In an embodiment, optical detection of fluorescently-tagged single molecules can be achieved by frequency-modulated absorption and laser-induced fluorescence. Fluorescence can be more sensitive because it is intrinsically amplified as each fluorophore emits thousands to perhaps a million photons before it is photo-bleached. Fluorescence emission usually occurs in a four-step cycle: 1) electronic transition from the ground-electronic state to an excited-electronic state, the rate of which is a linear function of excitation power, b) internal relaxation in the excited-electronic state, c) radiative or non-radiative decay from the excited state to the ground state as determined by the excited state lifetime, and d) internal relaxation in the ground state. Single molecule fluorescence measurements are considered digital in nature because the measurement relies on a signal/no signal readout independent of the intensity of the signal.

The high dynamic-range biomolecule quantification methods of the invention allow the measurement of over 10,000 biomolecules from a biological sample. The method can quantify biomolecules with concentrations from about 1 ag/mL to about 50 mg/mL and produce a dynamic range of more than 10¹⁰. The optical signals are digitized, and biomolecules are identified based on a code (ID code) of digital signals for each analyte.

As described above, in certain embodiments, target biomolecules are immobilized on a substrate, and probes are bound to the target biomolecule. Each of the probes comprises a detectable marker and specifically binds to a target biomolecule. In some embodiments, the detectable markers are fluorescent molecules that emit the same fluorescent color, and the signals for additional fluors are detected at each subsequent pass. During a pass, a set of probes comprising detectable markers are contacted with the substrate allowing them to bind to their targets. An image of the substrate is captured, and the detectable signals are analyzed from the image obtained after each pass. The information about the presence and/or absence of detectable signals is recorded for each detected position (e.g., analyte/target biomolecule) on the substrate.

In some embodiments, the invention comprises methods that include steps for detecting optical signals emitted from the probes comprising tags, counting the signals emitted during multiple passes and/or multiple cycles at various positions on the substrate, and analyzing the signals as digital information using a K-bit based calculation to identify each target biomolecule on the substrate. Error correction can be used to account for errors in the optically-detected signals, as described below.

In some embodiments, a substrate is bound with target biomolecules comprising N target biomolecules. To detect N target biomolecules, M cycles of probe binding and signal detection are chosen. Each of the M cycles includes 1 or more passes, and each pass includes N sets of probes, such that each set of probes specifically binds to one of the N target biomolecules. In certain embodiments, there are N sets of probes for the N target biomolecules.

In each cycle, there is a predetermined order for introducing the sets of probes for each pass. In some embodiments, the predetermined order for the sets of probes is a randomized order. In other embodiments, the predetermined order for the sets of probes is a non-randomized order. In one embodiment, the non-random order can be chosen by a computer processor. The predetermined order is represented in a key for each target biomolecule. A key is generated that includes the order of the sets of probes, and the order of the probes is digitized in a code to identify each of the target analytes/target biomolecules.

In some embodiments, each probe or probe set is associated with a distinct tag for detecting the target analyte/target biomolecule, and the number of distinct tags is less than the number of N target biomolecules. In that case, each N target biomolecule is matched with a sequence of M tags for the M cycles. The ordered sequence of tags is associated with the target biomolecule as an identifying code.

Devices and Techniques for Single Molecule Detection

Optical detection requires an optical detection instrument or reader to detect the signal from the labeled probes. U.S. Pat. No. 8,428,454 and U.S. Pat. No. 8,175,452, which are incorporated by reference in their entireties, describe exemplary imaging systems that can be used and methods to improve the systems to achieve sub-pixel alignment tolerances. In some embodiments, methods of aptamer-based microarray technology can be used. See Optimization of Aptamer Microarray Technology for Multiple Protein Targets, Analytica Chimica Acta 564 (2006).

Optical detection can be accomplished by detection of fluorescent or luminescent tags, described in more detail below and in U.S. Patent publication US20150330974 A1 which is incorporated herein by reference in its entirety.

Signal Analysis

After the detection process, the signals from each probe pool are counted, and the presence or absence of a signal and the color of the signal can be recorded for each position on the substrate.

From the detectable signals, K bits of information are obtained in each of M cycles for the N distinct target analytes/target biomolecules. The K bits of information are used to determine L total bits of information, such that K×M=L bits of information and L≥log₂ (N). The L bits of information are used to determine the identity (and presence) of N distinct target analytes/target biomolecules. If only one cycle (M=1) is performed, then K×1=L. However, multiple cycles (M>1) can be performed to generate more total bits of information L per analyte/target biomolecule. Each subsequent cycle provides additional optical signal information that is used to identify the target analyte/target biomolecule.

In practice, errors in the signals occur, and this confounds the accuracy of the identification of target analytes/target biomolecules. For instance, probes may bind the wrong targets (e.g., false positives) or fail to bind the correct targets (e.g., false negatives). Methods are provided, as described below, to account for errors in optical and electrical signal detection.

In certain aspects, the cell identification assay and/or the target identification assay comprises determining L total bits of information such that L is sufficient to reduce a false positive error rate of detection to less than 1 in 10⁶. In certain aspects, the false-positive detection rate is less than less than 1 in 10⁴, 1 in 10⁵, less than 1 in 10⁷, less than 1 in 10⁸ or less than 1 in 10⁹. In an aspect, L is a function of the misidentification rate for a target biomolecule at each cycle. In an aspect, the misidentification rate comprises the non-binding rate and the false binding rate of the probe to the target biomolecule. In certain aspects, L comprises bits of information that are ordered in a predetermined order. In certain aspects, the predetermined order is a random order. In certain aspects, L comprises bits of information comprising a key for decoding an order of the plurality of ordered target detection probe set and/or cell identifier probe set. In certain aspects, at least K bits of information comprise information about the absence of a signal for one of the N distinct target biomolecules.

The target detection probes and cell identifier probes used to detect the analytes/target biomolecules and cell identity, respectively, are introduced to the substrate in an ordered manner in each cycle. A key is generated that encodes information about the order of the probes for each target analyte/target biomolecule. The signals detected for each biomolecule can be digitized into bits of information. The order of the signals provides a code for identifying each analyte/target biomolecule and/or cell of origin, which can be encoded in bits of information.

In certain aspects the cell identifier probes and/or target detection probes have a cross-reactivity with non-target biomolecule of greater than 2%, 5%, 10%, 15%, 20%, or 25%. In certain aspects, at least one of the target biomolecules does not bind to a corresponding cell identifier probe and/or target detection probe for at least 10%, at least 20%, at least 30%, or at least 40% of cycles.

Methods for Electronic Detection of Target Biomoleucles

In certain embodiments, electronic detection methods are used to determine the presence or absence of target biomolecules in a sample. In certain embodiments, the methods for electronic detection comprise using ion sensitive field effect transistors (ISFET) which measures hydrogen ion concentrations in solution. ISFETs are described in further detail in U.S. Pat. No. 7,948,015, filed on Dec. 14, 2007, to Rothberg et al., and U.S. Publication No. 2010/0301398, filed on May 29, 2009, to Rothberg et al., which are both incorporated by reference in their entireties.

In certain embodiments, electrical detection is accomplished using ISFET integrated with MEMS (micro-electrical mechanical systems) structures for enhanced sensitivity. Techniques include use of poly-A tags with and without differential stops, complementary specific and non-specific probes for detailed characterization of target biomolecules, highly multiplexed single molecule identification and quantification using probes.

In certain embodiments, target biomolecules are tagged with oligonucleotide tail regions and the oligonucleotide tags are detected using ISFETS. ISFETs present a sensitive and specific electrical detection system for the identification and characterization of target biomolecules. In an embodiment, the electrical detection methods disclosed herein are carried out by a computer (e.g., a processor). The ionic concentration of a solution can be converted to a logarithmic electrical potential by an electrode of an ISFET, and the electrical output signal can be detected and measured.

In an embodiment, an ISFET is used to detect a tail region of a probe or tag and then to identify the corresponding target biomolecule or cell of origin. For example, a target biomolecule can be immobilized on a substrate, such as an integrated-circuit chip that contains one or more ISFETs. When the corresponding probe (e.g., aptamer and tail region) is added and specifically binds to the target biomolecule, nucleotides and enzymes (polymerase) are added for transcription of the tail region. The ISFET detects the release hydrogen ions as electrical output signals and measures the change in ion concentration when the dNTP's are incorporated into the tail region. The amount of hydrogen ions released corresponds to the lengths and stops of the tail region, and this information about the tail regions can be used to differentiate among various tags.

The simplest type of tail region is one composed entirely of one homopolymeric base region. In this case, there are four possible tail regions: a poly-A tail, a poly-C tail, a poly-G tail, and a poly-T tail. However, it is often desirable to have a great diversity in tail regions.

A method of generating diversity in tail regions is by providing stop bases within a homopolymeric base region of a tail region. A stop base is a portion of a tail region comprising at least one nucleotide adjacent to a homopolymeric base region, such that the at least one nucleotide is composed of a base that is distinct from the bases within the homopolymeric base region. In an embodiment, the stop base is one nucleotide. In other embodiments, the stop base comprises a plurality of nucleotides. Generally, the stop base is flanked by two homopolymeric base regions. In an embodiment, the two homopolymeric base regions flanking a stop base are composed of the same base. In another embodiment, the two homopolymeric base regions are composed of two different bases. In another embodiment, the tail region contains more than one stop base.

In an example, an ISFET can detect a minimum threshold number of 100 hydrogen ions. Target Biomoleculel is bound to a composition with a tail region composed of a 100-nucleotide poly-A tail, followed by one cytosine base, followed by another 100-nucleotide poly-A tail, for a tail region length total of 201 nucleotides. Target Biomolecule2 is bound to a composition with a tail region composed of a 200-nucleotide poly-A tail. Upon the addition of dTTPs and under conditions conducive to polynucleotide synthesis, synthesis on the tail region associated with Target Biomoleculel will release 100 hydrogen ions, which can be distinguished from polynucleotide synthesis on the tail region associated with Target Biomolecule2, which will release 200 hydrogen ions. The ISFET will detect a different electrical output signal for each tail region. Furthermore, if dGTPs are added, followed by more dTTPs, the tail region associated with Target Biomoleculel will then release one, then 100 more hydrogen ions due to further polynucleotide synthesis. The distinct electrical output signals generated from the addition of specific nucleoside triphosphates based on tail region compositions allow the ISFET to detect hydrogen ions from each of the tail regions, and that information can be used to identify the tail regions and their corresponding target analytes.

Various lengths of the homopolymeric base regions, stop bases, and combinations thereof can be used to uniquely tag each biomolecule in a sample. Additional description about electrical detection of aptamers and tail regions to identify target biomolecules in a substrate are described in U.S. Provisional Application No. 61/868,988, which is incorporated by reference in its entirety.

In other embodiments, antibodies are used as probes in the electrical detection method described above. The antibodies may be primary or secondary antibodies that bind via a linker region to an oligonucleotide tail region that acts as tag.

These electrical detection methods can be used for the simultaneous detection of hundreds (or even thousands) of distinct target biomolecules. Each target biomolecule can be associated with a digital identifier, such that the number of distinct digital identifiers is proportional to the number of distinct target biomolecules in a sample. The identifier may be represented by a number of bits of digital information and is encoded within an ordered tail region set. Each tail region in an ordered tail region set is sequentially made to specifically bind a linker region of a probe region that is specifically bound to the target biomolecule.

Alternatively, if the tail regions are covalently bonded to their corresponding probe regions, each tail region in an ordered tail region set is sequentially made to specifically bind a target biomolecule.

In an embodiment, one cycle is represented by a binding and stripping of a tail region to a linker region, such that polynucleotide synthesis occurs and releases hydrogen ions, which are detected as an electrical output signal. Thus, the number of cycles for the identification of a target biomolecule is equal to the number of tail regions in an ordered tail region set. The number of tail regions in an ordered tail region set is dependent on the number of target biomolecules to be identified, as well as the total number of bits of information to be generated. In another embodiment, one cycle is represented by a tail region covalently bonded to a probe region specifically binding and being stripped from the target biomolecule.

The electrical output signal detected from each cycle is digitized into bits of information, so that after all cycles have been performed to bind each tail region to its corresponding linker region, the total bits of obtained digital information can be used to identify and characterize the target biomolecule in question. The total number of bits is dependent on a number of identification bits for identification of the target biomolecule, plus a number of bits for error correction. The number of bits for error correction is selected based on the desired robustness and accuracy of the electrical output signal. Generally, the number of error correction bits will be 2 or 3 times the number of identification bits.

Error-Correction Methods

In optical detection methods described above, errors can occur in binding and/or detection of signals. Method for error-correction are described in detail in U.S. Patent publication US20150330974 A1, which is incorporated herein by reference in its entirety.

In some cases, the error rate can be as high as one in five (e.g., one out of five fluorescent signals is incorrect). This equates to one error in every five-cycle sequence. Actual error rates may not be as high as 20%, but error rates of a few percent are possible. In general, the error rate depends on many factors including the type of analytes/target biomolecules in the sample and the type of probes used. In an optical detection method, a probe may not bind to its target or bind to the wrong target.

Additional cycles are generated to account for errors in the detected signals and to obtain additional bits of information, such as parity bits. The additional bits of information are used to correct errors using an error correcting code. In an embodiment, the error correcting code is a Reed-Solomon code, which is a non-binary cyclic code used to detect and correct errors in a system. In other embodiments, various other error correcting codes can be used. Other error correcting codes include, for example, block codes, convolution codes, Monte Carlo codes, Golay codes, Hamming codes, BCH codes, AN codes, Reed-Muller codes, Goppa codes, Hadamard codes, Walsh codes, Hagelbarger codes, polar codes, repetition codes, repeat-accumulate codes, erasure codes, online codes, group codes, expander codes, constant-weight codes, tornado codes, low-density parity check codes, maximum distance codes, burst error codes, luby transform codes, fountain codes, and raptor codes. See Error Control Coding, 2^{nd} Ed., S. Lin and DJ Costello, Prentice Hall, New York, 2004.

In certain embodiments, error correction can reduce the false-positive detection rate to less than 1 in 10⁴, less than 1 in 10⁵, less than 1 in 10⁷, less than 1 in 10⁸ or less than 1 in 10⁹.

Methods of Detecting Oncoproteins or Oligonucleotides from Single Cells from Blood Samples

The methods described herein can be used to perform a Cell Identification Assay and Target Identification Assay to determine a cellular source of a tumor associated biomolecule (e.g., an oncoprotein or tumor associated oligonucleotide).

For example, a Cell Identification Assay and Target Identification Assay can be performed to detect oncoproteins or tumor associated oligonucleotide associated with individual circulating tumor cells (CTCs). In an embodiment, blood samples are collected from a patient suspected of having cancer. In some embodiments, the CTCs are isolated and enriched. CTCs can be isolated and enriched by methods known in the art such as, but not limited to, enrichment by gradient centrifugation, filtration through polycarbonate membranes, antibody-based enrichment methods (such as magnetic activated cell sorting or other similar methods comprising use of antibodies and particles with magnetic surfaces) and microfluidic devices. In some embodiments, the isolated and enriched cells are then resuspended in an appropriate buffer (such as phosphate buffered saline) and isolated as single cells. Isolation/enrichment and resuspension can be performed using a FACS based methods to distribute single cell into individual wells (e.g., of a 384 well plate). An appropriate volume of lysis buffer comprising detergent can be added to wells to lyse plasma and nuclear membrane, releasing cellular material comprising target biomolecules from each cells. Methods of sample preparation, including isolation and lysis of cells, can be performed using several methods known in the art.

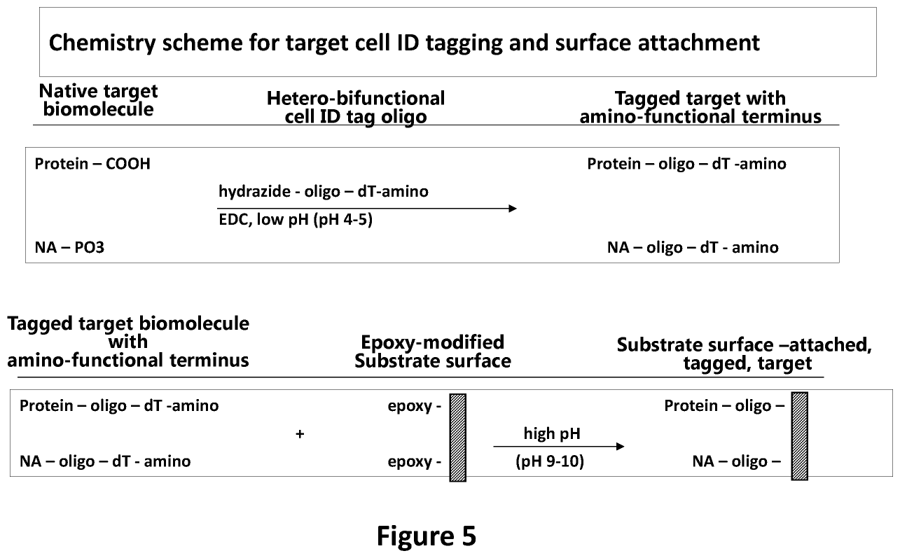

Cell identifier tags comprising cell identifier oligonucleotide barcodes (e.g., oligonucleotide barcodes 15 nucleotides in length) can be conjugated to nucleic acid and proteins in each well. A distinct barcode can be added to each well, such that a cell identifier tag is specific to a single cell or a population of cells in a well. Cell identifier oligonucleotide tags can be conjugated to proteins and nucleic acids using mono-functional or hetero-bifunctional modified oligonucleotides. In an example, a mixture of protein and nucleic acids can be conjugated to a common hydrazide modified tag oligonucleotide, simultaneously, in the same mix, such as by using carbodiimide activation chemistry (FIG. 5). In one aspect, 5′-hydrazide modified oligonucleotides can be conjugated to a protein through native carboxyl functionality on the protein using a water soluble carbodiimide to activate the protein carboxyl towards reaction with the hydrazide moiety of the oligonucleotide. Conjugation of an oligonucleotide through a protein carboxyl functionality leaves native primary amine functionality (lysine residues) for attachment to detection platform supports. In another aspect, hydrazide modified oligonucleotides can also be conjugated to nucleic acids through native terminal 5′-phosphate functionality using a water soluble carbodiimide. Hetero-bifunctional oligo modifications can allow, for example, conjugation and attachment to a detection platform support. In another aspect, a 5′-hydrazide tag oligo modification along with a 3′-dT-amino modification can allow, for example, conjugation of the tag to a target nucleic acid through the hydrazide moiety while leaving a primary amine moiety on the tag for subsequent attachment to a detection platform support, e.g., an epoxy modified surface. A hetero-bifunctional tag strategy for nucleic acids can also be compatible with proteins, and can allow, for example, a mixture of protein and nucleic acids to be conjugated to a common tag simultaneously, and subsequently attached to a support.

Tagged nucleic acid and proteins can be pooled and distributed on a solid support, such as an epoxy modified solid support. Each nucleic acid and protein can be immobilized at a distinct location on a support. A 3′-dT-amino modification on a nucleic acid and protein can allow, for example, attachment to an epoxy surface under high pH conditions (e.g., pH 9-10) (FIG. 5). A support comprising bound tagged nucleic acids and proteins can be contacted with target barcode probes comprising a plurality of distinct target barcode probes, such as antibodies which specifically bind to an oncoprotein of interest (e.g., antibodies specific for K-RasV12, c-Myc, EGFR, PDGFR, Raf and Erk), as well as oligonucleotides complementary to nucleic acid comprising sequences for activating mutations (e.g., K-RasV12, c-Myc, EGFR, PDGFR, Raf and Erk). Target barcode probes comprising antibodies can be conjugated to a target barcode probe oligonucleotide (e.g., 20 a target barcode probe oligonucleotide nucleotides in length) that is distinct for each type of antibody.

A cell identification assay can be performed comprising addition of cell identifier probes wherein each cell identifier probe is bound to a cell identifier probe oligonucleotide and a fluorophore corresponding to the cell identifier probe oligonucleotide sequence. A cell identifier probe oligonucleotide can be hybridized to a cell identifier tag (cell identifier oligonucleotide barcode). A number of detection cycles (e.g., M=10) can be performed to identify a cell identifier probe bound to an immobilized biomolecule on a support (i.e., a substrate), wherein each cycle comprises contacting the support with cell identifier probes corresponding to a single cell, e.g., a CTC, washing the support to remove unbound cell identifier probes, and detecting a fluorescence at each region on the support using an optical imaging system. In some embodiments, to prepare for subsequent identification cycles, a cycle can further comprise denaturing a cell identifier probes from a support. Detection cycles can use ordered probe reagent sets designed to provide signals that can be used for cell identifier tag identification and for error correction, as described in U.S. Patent Publication 2015/0330974, incorporated by reference.

Analysis of color codes for identification of sequences can be performed using a single-color imaging system. For example, an imaging system can measure a single color image for a first cycle, where A and B molecules fluoresce, but C and D are dark (no probes and no signal). In some examples, probes for targets A and B can be stripped. In some examples, a second cycle can be performed and antibody probes for targets C and D can be introduced and imaged, and then antibody probes for C and D may be stripped. In some examples, a third cycle can be performed and antibody probes for targets A and C can be introduced and imaged, and then antibody probes for targets A and C may then stripped. In some examples, a fourth cycle can be performed and antibody probes for targets B and D can be introduced and imaged. After imaging, including after imaging multiple cycles, an ID (code of fluorescent signals) for a target molecule at each position can be determined. In some embodiments, the number of imaging cycles performed is sufficient to determine an ID for a target molecule at each position. Mapping of target biomolecules to a cell of origin, e.g. an individual CTC, to a color sequence can be performed such that each color corresponds to a cell identifier tag sequence, which maps to 1 or 0 with 1 bit of information being acquired per cycle.

A Target Identification Assay can be performed comprising addition of a target detection probe set comprising a plurality of target detection probes. Each target detection probe can comprise a target detection probe oligonucleotide and a target identification detectable marker. A target identification detectable maker can include fluorophores corresponding to each type of antibody a target detection probe binds preferentially. A target detection probe oligonucleotide can anneal preferentially to a complementary target identifier oligonucleotide barcode. A number of detection cycles (e.g., N=10) can be performed to identify the target biomolecules (e.g., oncoproteins) at each location on a support. Each cycle can comprise contacting a support with target detection probes corresponding to an individual oncoprotein, washing the support to remove unbound target detection probe, and detecting a target identification detectable maker (e.g., fluorescence) at each region on the array. In some embodiments, to prepare for subsequent identification cycles, a cycle can further comprise denaturing a target detection probes from a support. Detection cycles can use ordered probe reagent sets designed to provide signals that can be used for target identification and for error correction, as described in U.S. Patent Publication 2015/0330974, incorporated by reference. Analysis of color codes for identification of sequences can be performed using a two-color imaging system. Mapping of target oncoproteins to a color sequence can be performed such that each color corresponds to a target barcode probe (e.g., an antibody specific for an oncoprotein), which maps to 1 or 0 with 1 bit of information being acquired per cycle. Methods of detection, including cycled detection using ordered probe sets and detection error reduction, instrumentation for detection and data analysis are described in detail in U.S. Patent Publication 2015/0330974 A1, and International PCT Publication WO 2014/078855A1, both of which are incorporated herein by reference in their entirety

Mapping results of a Cell Identification Assay and Target Identification Assay described above can be advantageously used, for example, to identify a cellular source of a tumor associated biomolecule, such as spatially mapping which oncoproteins or tumor associated oligonucleotides identified during the Target Identification Assay correspond with which specific CTCs identified during the Cell Identification Assay, with high sensitivity and specificity.

### REFERENCES AND OTHER EMBODIMENTS

All references, issued patents and patent applications cited within the body of the instant specification are hereby incorporated by reference in their entirety, for all purposes.

While the invention has been particularly shown and described with reference to a preferred embodiment and various alternate embodiments, it will be understood by persons skilled in the relevant art that various changes in form and details can be made therein without departing from the spirit and scope of the invention.

1. A method of detecting the presence or absence of one or more target biomolecules from a single cell suspected of being present in a sample comprising:
obtaining a sample comprising a plurality of cells suspected of comprising one or more target biomolecules;
isolating single cells from the plurality of cells into individual compartments;
lysing the single cells to yield cellular material comprising a plurality of biomolecules;
binding at least one cell identifier tag to the plurality of biomolecules from each single cell, wherein the cell identifier tag is unique for each isolated single cell;
distributing the plurality of biomolecules from the plurality of isolated cells onto a substrate such that the plurality of biomolecules are immobilized on the substrate at spatially separate regions;
performing a cell identification assay to determine the cellular source for each of the plurality of immobilized biomolecules at the spatially separate regions; and
performing a target identification assay to identify the presence or absence of the one or more target biomolecules at the spatially separate regions.

2. The method of claim 1, wherein performing the cell identification assay comprises:
contacting the substrate comprising the immobilized plurality of biomolecules with a cell identifier probe set, wherein the cell identifier probe set comprises a plurality of cell identifier probes each comprising a cell identification detectable marker, wherein each cell identifier probe binds preferentially to at least one cell identifier tag specific for each of the isolated single cells;
removing unbound cell identifier probes from the surface of the substrate; and
detecting the presence or absence of a signal from the cell identification detectable marker at the spatially separate regions.

3. The method of claim 1, wherein performing the cell identification assay comprises:
performing at least M detection cycles to generate a cell identification signal detection sequence for at least one of the spatially separate regions, wherein M is at least two, each cycle comprising:
contacting the substrate comprising the immobilized plurality of biomolecules with a cell identifier probe set, wherein the cell identifier probe set comprises a plurality of cell identifier probes comprising a cell identification detectable marker, wherein each of the cell identifier probe binds preferentially to at least one cell identifier tag specific for each of the isolated single cells;
removing unbound cell identifier probes from the surface of the substrate;
detecting the presence or absence of a signal from the cell identification detectable marker at the spatially separate regions; and
if the cycle number is less than M, removing bound cell identifier probes from the substrate.

4. The method of claim 3, further comprising analyzing the cell identification signal detection sequence generated by the M cycles at at least one of the spatially separate regions to determine the cellular origin of the immobilized biomolecule.

5. The method of any one of the above claims, wherein performing the target identification assay comprises:
contacting the substrate comprising the immobilized plurality of biomolecules with a target detection probe set, wherein the target detection probe set comprises a plurality of target detection probes that each bind preferentially to at least one of the one or more target biomolecules, the plurality of target detection probes each comprising a target identification detectable marker;
removing unbound target detection probes from the surface of the substrate; and
detecting the presence or absence of a signal from the target identification detectable marker at the spatially separate regions.

6. The method of any one of claims 1-4, wherein performing the target identification assay comprises:
performing at least N detection cycles to generate a target identification signal detection sequence for at least one of the spatially separate regions, wherein N is at least two, each cycle comprising:
contacting the substrate comprising the immobilized plurality of biomolecules with a target detection probe set, wherein the target detection probe set comprises a plurality of target detection probes that each directly or indirectly bind preferentially to at least one of the one or more target biomolecules, the plurality of target detection probes each comprising a target identification detectable marker;
removing unbound target detection probes from the surface of the substrate;
detecting the presence or absence of a signal from the target identification detectable marker at the spatially separate regions; and
if the cycle number is less than N, removing bound target detection probes from the substrate.

7. The method of claim 6, further comprising analyzing the target identification signal detection sequence generated by the N cycles at at least one of the spatially separate regions to determine the presence or absence of the one or more target biomolecules.

8. The method of any one of the above claims, comprising analyzing the signal from one or more of the spatially separate regions from the cell identification assay and the target identification assay to determine the presence or absence of the one or more target biomolecules in one or more of the plurality of single cells.

9. The method of any one of the above claims, further comprising determining the presence or absence of a plurality of the one or more target biomolecules from one of the plurality of cells.

10. The method of any one of claims 5-10, wherein the method further comprises contacting the cellular material with a target barcode probe comprising a target identification tag, wherein the target barcode probe preferentially binds at least one of the one or more target biomolecules, and wherein at least one of the target detection probes binds preferentially to the target identification tag.

11. The method of claim 10, wherein the target barcode probe comprises an antibody.

12. The method of claim 10, wherein binding of the target barcode probe to the target biomolecule is performed using a linker or adapter molecule.

13. The method of any one of claim 10 or 11, wherein the target identification tag comprises a target identifier oligonucleotide barcode.

14. The method of claim 13, wherein the target detection probe comprises a target detection probe oligonucleotide, and wherein the target identifier oligonucleotide barcode comprises a sequence complementary to the target detection probe oligonucleotide.

15. The method of any one of claims 2-14, wherein the cell identifier probe binds specifically to one or more of the at least one cell identifier tags.

16. The method of any one of claims 5-15, wherein the target detection probe binds specifically to one or more of the at least one target biomolecules.

17. The method of any one of the above claims, wherein the cell identification assay and the target identification assay are performed sequentially at each of the spatially separate regions on the substrate.

18. The method of any one of the above claims, wherein the cellular material comprises protein, DNA, RNA, or combinations thereof.

19. The method of any one of the above claims, wherein the cell identifier tag comprises a cell identifier oligonucleotide barcode.

20. The method of claim 19, wherein the cell identifier probe comprises a cell identifier probe oligonucleotide.

21. The method of claim 20, wherein the cell identifier probe oligonucleotide comprises a sequence complementary to the cell identifier oligonucleotide barcode.

22. The method of any one of claims 5-21, wherein the target detection probe comprises an antibody.

23. The method of any one of claims 2-22, wherein the cell identification detectable marker comprises a fluorescent tag.

24. The method of any one of claims 5-23, wherein the target identification detectable marker comprises a fluorescent tag.

25. The method of any one of the above claims, wherein the target biomolecule is protein.

26. The method of claim 25, wherein the protein is created by ribosome display.

27. The method of any one of the above claims, wherein the target biomolecule is nucleic acid.

28. The method of any one of the above claims, wherein binding of the cell identifier tag to the plurality of biomarkers is performed using a linker or adapter molecule.

29. The method of any one of claims 2-28, wherein binding of the cell identifier tag to the plurality of biomolecules is performed by enzymatic conjugation.

30. The method of any one claims 2-29, wherein the cell identifier probe comprises a linker or adapter molecule bound to the cell identification detectable marker.

31. The method of any one claims 5-30, wherein the target detection probe comprises a linker or adapter molecule bound to the target identification detectable marker.

32. The method of any one of the above claims, wherein the sample comprises cells derived from an individual.

33. The method of claim 32, wherein the cells are from tissue derived from a biopsy.

34. The method of claim 33, wherein the biopsy is a tumor biopsy.

35. The method of any of the above claims, wherein the sample is suspected of comprising one or more cancer cells.

36. The method of claim 32, wherein the cells are circulating cells derived from the blood or plasma of the individual.

37. The method of any one of the above claims, wherein the target identification assay comprises determining L total bits of information such that L is sufficient to reduce a misidentification error rate of detection to less than 1 in 10², 1 in 10³, 1 in 10⁴, 1 in 10⁵, 1 in 10⁶, 1 in 10⁷, or 1 in 10⁸.

38. The method of claim 37, wherein the misidentification error rate comprises false positives, false negatives, or both.

39. The method of any one of the above claims, wherein the method comprises determining a quantity of the one or more target biomolecules from one or more of the plurality of cells.

40. The method of any one of the above claims, wherein the method comprises identifying at least one sub-population of cells, comprising at least one cell, within the sample.

41. A method of preparing a sample for single cell analysis, comprising:
obtaining a sample comprising a plurality of cells suspected of comprising one or more target biomolecules;
isolating single cells from the plurality of cells into individual compartments;
lysing the single cells to yield cellular material comprising a plurality of biomolecules; and
binding at least one cell identifier tag to the plurality of biomolecules from each single cell, and wherein the cell identifier tag is unique for each isolated single cell.

42. The method of any one of claims 1-41, wherein the isolation of the single cells from the plurality of cells is performed using a microfluidic device.

43. A method of detecting the presence or absence of one or more target biomolecules from a single cell suspected of being present in a sample comprising:
obtaining a sample derived from a plurality of isolated cells suspected of comprising one or more target biomolecules, the sample comprising a plurality of biomolecules bound to at least one cell identifier tag unique for each one of the plurality of isolated cells;
distributing the plurality of biomolecules from the plurality of isolated cells onto a substrate such that the plurality of biomolecules are immobilized on the substrate at spatially separate regions;
performing a cell identification assay to determine the cellular source for each of the plurality of immobilized biomolecules at the spatially separate regions; and
performing a target identification assay to identify the presence or absence of the one or more target biomolecules at the spatially separate regions.

44. The method of any one of the above claims, wherein the plurality of target biomolecules are pooled prior to the distributing.
